(19) United States
(12) Patent Application Publication
(10) Pub. No.: US 2025/0179403 A1
(43) Pub. Date: Jun. 5, 2025

(54) A CELL PROCESSING SYSTEM FOR HOUSING A BIOREACTOR

(71) Applicant: ORIBIOTECH LTD, London, Greater London (GB)

(72) Inventors: Stuart Milne, Cambridge (GB); Mark Varley, Cambridge (GB); William Raimes, London (GB); Stephen Shapka, Benson (GB); Farlan Veraitch, London (GB); Liam Pegram, London (GB); Julian Brandon-Jones, Cambridge (GB)

(21) Appl. No.: 18/844,017

(22) PCT Filed: Mar. 2, 2023
(86) PCT No.: PCT/GB2023/050490

§ 371 Date: Sep. 4, 2024

(30) Foreign Application Priority Data
Mar. 9, 2022 (GB) 2203270.0

(51) Int. Cl. C12M 3/00; C12M 1/00; C12M 1/34

(52) U.S. Cl. CPC C12M 23/48; C12M 23/26; C12M 23/38; C12M 41/40

(57) ABSTRACT

The present application provides a cell processing system for housing a bioreactor and methods associated therewith. The cell processing system is adapted to support the bioreactor. The cell processing system includes a load sensing unit operable to detect a weight of the bioreactor.

### CROSS-REFERENCE TO RELATED APPLICATIONS

This application is a national phase entry under 35 U.S.C. § 371 of International Patent Application PCT/GB2023/050490, filed Mar. 2, 2023, designating the United States of America and published as International Patent Publication WO 2023/170387 A1 on Sep. 14, 2023, which claims the benefit under Article 8 of the Patent Cooperation Treaty of Great Britain Patent Application Serial No. 2203270.0, filed Mar. 9, 2022.

### TECHNICAL FIELD

This disclosure relates to a cell processing system for housing a bioreactor, and particularly to a cell processing system that includes a load sensing unit.

### BACKGROUND

Cell and gene therapy manufacturing processes are often complex and include manual or semi-automated steps across several devices. Equipment systems used in various steps, or unit operations, of cell-based therapeutic products (CTP) manufacturing may include devices for various functions. These various functions may be, for example, cell collection, cell isolation, cell selection, cell expansion, cell washing, volume reduction, cell storage or transportation. The unit operations can vary immensely based on the manufacturing model (i.e., autologous versus allogenic), cell type, intended purpose, among other factors. In addition, cells are “living” entities sensitive to even the simplest manipulations, for example, such as differences in a cell transferring procedure. The role of cell manufacturing equipment in ensuring scalability and reproducibility is an important factor for cell and gene therapy manufacturing.

In addition, cell-based therapeutic products (CTP) have gained significant momentum thus there is a need for improved cell manufacturing equipment for various cell manufacturing procedures. These manufacturing procedures, may include, for example, stem cell enrichment, generation of chimeric antigen receptor (CAR) T cells, and various cell manufacturing processes such as collection, purification, gene modification, incubation, recovery, washing, infusion into a patient, or freezing.

The culture or processing of cells typically requires the use of a device to hold the cells, for example, in an appropriate culture medium when culturing the cells. The known devices include shaker flasks, roller bottles, T-flasks, bags and the like. Such devices are typically required to be connected to other devices, such as containers, interfaces or the like, so that various media may be introduced to, or removed from, the device holding the cells. Typically, cells in a culture medium can be added to the device from a flexible bag that is attached using a connecting tube. Alternatively, cells can be transferred by a pipette or by a syringe.

### BRIEF SUMMARY

In accordance with the present disclosure there is provided a cell processing system for housing a bioreactor, the cell processing system being adapted to support the bioreactor and comprising a load sensing unit operable detect a weight of the bioreactor.

Advantageously, by providing a load sensing unit as a part of the cell processing system it is possible to detect the weight of the bioreactor without removing the bioreactor from the cell processing system. The weight of the bioreactor may be monitored or periodically checked, for example, before, after, and/or during adding or removing material from the bioreactor.

In examples, the cell processing system may further comprise a housing and a support that supports the bioreactor within the housing. The housing may provide a controlled environment. The controlled environment may be a sterile environment and can be sealed from the external environment. The housing may have one or more controlled parameters, for example, temperature, humidity, pressure, and/or gas concentrations (e.g., oxygen, carbon dioxide). Accordingly, it is advantageous to provide a cell processing system with a load sensing unit that can detect the weight of the bioreactor without having to remove the bioreactor from the housing.

In examples, the support is pivotally mounted to the housing and the load sensing unit comprises a load cell arranged such that a torque of the support is imparted onto the load cell. In this way, the load sensing unit can detect the weight of the bioreactor on the support.

In other examples, the load sensing unit is adapted to releasably connect to the bioreactor. The load sensing unit can connect to the bioreactor to detect the weight of the bioreactor, and can disconnect from the bioreactor when not detecting the weight to allow other operations to proceed.

In examples, during use the load sensing unit may be disposed above the bioreactor and operable to releasably connect to a top of the bioreactor.

In some examples, the load sensing unit is movable to lift the bioreactor from the support to a raised position in which the weight of the bioreactor is borne by the load sensing unit. A sensor (load cell) of the load sensing unit may then detect the weight of the bioreactor.

Additionally or alternatively, the support may be movable to disengage the bioreactor such that the weight of the bioreactor is borne by the load sensing unit. That is, the load sensing unit can be connected to the bioreactor and then the support can disengage such that the weight of the bioreactor is entirely borne by the load sensing unit. A sensor (load cell) of the load sensing unit may then detect the weight of the bioreactor.

In examples, the load sensing unit may comprise a coupling operable to releasably connect to the bioreactor.

In examples, the coupling is mounted to a platform that is movable relative to the bioreactor. The platform may be moveable in a vertical direction, toward and away from the top of the bioreactor. The coupling may be mounted to the platform via a load cell. In this way, when the bioreactor is coupled to the load sensing unit the weight of the bioreactor can be detected by lifting the bioreactor using the load sensing unit. Additionally or alternatively, the load cell can measure a compression force applied to the bioreactor by the load sensing unit.

In examples, the coupling comprises a clamp operable to releasably connect to the bioreactor. The clamp may comprise an actuator and at least one arm moveable by the actuator to clamp onto the bioreactor. The actuator may include an electric motor. The actuator may include a gear train operably disposed between the actuator and the clamp. The clamp may comprise a pair of opposing arms that are rotatable by the actuator (e.g., via the gear train) in opposite directions to connect to, and disconnect from, the bioreactor.

In examples, the load sensing unit further comprises one or more sensors arranged to detect the presence or proximity of the bioreactor to the clamp. Additionally or alternatively, the load sensing unit further comprises one or more sensors arranged to detect gripping of the bioreactor by the clamp.

In examples, the bioreactor comprises a clamping feature to which the clamp can connect. The clamping feature may comprise a lip or circular protrusion arranged to match a shape of the clamping arms.

In examples, the load sensing unit is disposed above the bioreactor within the housing, and the load sensing unit is operable to releasably connect to a top of the bioreactor. The top of the bioreactor may be a part of an expansion container on the bioreactor, or a part of a lid assembly. Preferably, the load sensing unit connects to the uppermost part of the bioreactor and the load sensing unit is generally positioned above the bioreactor within the housing.

In examples, the load sensing unit may comprise a pivotally mounted hinge plate. The hinge plate may be releasably connectable to the bioreactor such that the weight of the bioreactor imparts a torque on the hinge plate. The clamp may be disposed on the hinge plate. The load sensing unit may comprise a load cell arranged such that the torque imparted on the hinge plate is imparted onto the load cell. That is, the weight of the bioreactor acts on the hinge plate, which in turn imparts a force on the load cell. In some examples, the load cell is compressed by the torque of the hinge plate. In other examples, the load cell is placed under tension by the torque of the hinge plate. In each example, a strain gauge or other sensor of the load cell can detect the force, and a controller of the cell processing system can determine a weight of the bioreactor from the detected force.

In examples, the coupling may comprise a bayonet fitting. In examples, the bioreactor may comprise a first bayonet fitting and the load sensing unit may comprise a second bayonet fitting that is couplable to the first bayonet fitting. In examples, the cell processing system may comprise an actuator for rotating the bioreactor relative to the load sensing unit for coupling the first and second bayonet fittings.

In examples, the coupling of the load sensing unit comprises a bayonet fitting having a plurality of lugs arranged to engage a bayonet fitting of the bioreactor. The bayonet fitting of the bioreactor may have a plurality of recesses through which the lugs of the coupling can pass, a plurality of lugs. Relative rotation of the bioreactor and the coupling can align the lugs to couple the load sensing unit to the bioreactor.

In other examples, the load sensing unit comprises a bayonet fitting having a plurality of grooves formed on an outer surface of a body, for example, a cylindrical body. The bioreactor includes an opening having a plurality of pins extending inwardly into the opening. The pins engage the grooves to provide a bayonet coupling. The grooves include one or more traps in which the pins are received when the coupling is coupled to the bioreactor. A first trap may engage the pin when the load sensing unit presses down on the bioreactor, and a second trap may engage the pin when the load sensing unit lifts up the bioreactor.

In examples, the coupling may comprise at least one spring arm (a spring-biased arm). In examples, the load sensing unit may comprise a plurality of spring arms arranged to engage an edge of an opening in the bioreactor to couple the load sensing unit to the bioreactor. In examples, each of the plurality of spring arms may comprise a notch arranged to engage the edge of the opening of in the bioreactor. The notches may be shaped such that the spring arms are deflected inwards as the load sensing is moved toward the bioreactor and spring outwards as the notches align with the edge of the opening. The notches may be shaped such that the load sensing unit can be decoupled from the bioreactor by moving the load sensing unit away from the bioreactor to deflect the spring arms. The or each spring arm may be spring-biased by a torsional spring, a helical spring or a like resiliently deformable member.

In examples, the load sensing unit may further comprise one or more sensors arranged to detect the presence or proximity of the bioreactor to the coupling. Additionally or alternatively, the load sensing unit further comprises one or more sensors arranged to detect coupling of the load sensing unit and the bioreactor.

In some examples, the bioreactor comprises a compressible element. For example, the bioreactor may comprise a compressible container defining the main volume of the bioreactor, and/or a compressible expansion container in fluid communication with the main volume of the bioreactor. In such examples, the load sensing unit may be adapted to detect a compression force as the compressible element is compressed. An actuator may act to compress the compressible element, and a compression force can be detected by the load sensing unit.

In some examples, the load sensing unit comprises an actuator operable to the compress the compressible element. The actuator may be operable to either lift the bioreactor to detect the weight of the bioreactor (as described above), or to compress the compressible element. In examples, the cell processing system may further comprise a sensor configured to detect a displacement of the compressible element as the compressible element is compressed.

By detecting the compression force and/or displacement of the compressible element it is possible to determine if the bioreactor has a leak. In particular, if during compression a predetermined compressive force threshold is reached then it may be assumed that the bioreactor does not have a leak. Alternatively, a pre-determined compressive force may be applied to the compressible element of the bioreactor and the displacement monitored over time to detect signs of a leak.

In examples, the cell processing system described above may further comprise a bioreactor. The bioreactor may comprise a container and a lid assembly having one or more connector interfaces for accessing the internal volume of the container. As mentioned above, the bioreactor may include a compressible element. The container of the bioreactor may be a compressible element, for example, having a compressible or collapsible side wall (e.g., bellows wall). In some examples the bioreactor, in particular, the lid assembly, may include an expansion container that may be a compressible element. In particular, the expansion container may be compressible, having a compressible or collapsible side wall (e.g., bellows wall).

In accordance with the present disclosure there is also provided a method of detecting a weight of a bioreactor within a cell processing system adapted to support the bioreactor. The method comprises bearing the weight of the bioreactor on a load sensing unit of the cell processing system, and determining a weight of the bioreactor based on a load detected by the load sensing unit.

Advantageously, the load sensing unit is disposed within a housing of the cell processing system such that the bioreactor does not need to be removed from the cell processing system to detect its weight.

In examples, the method comprises releasably connecting the load sensing unit to the bioreactor. For example, the method may comprise connecting the load sensing unit to a lid assembly of the bioreactor.

In examples, the method comprises moving the load sensing unit to lift the bioreactor from a support of the cell processing system. By lifting the bioreactor from the support the weight of the bioreactor is borne by the load sensing unit. A sensor, e.g., a load cell, can then detect the weight of the bioreactor.

Additionally or alternatively, if the bioreactor comprises a compressible element then the method may comprise moving the load sensing unit to compress the compressible element. In such examples the method may further comprise detecting a compression force as the load sensing unit compresses the compressible element of the bioreactor. Additionally or alternatively, the method may comprise detecting a displacement of the load sensing unit as the load sensing unit compresses the compressible element of the bioreactor. By detecting the compressive force and/or displacement it is possible to leak-test the bioreactor.

In accordance with the present disclosure there is also provided a method of detecting a leak in a bioreactor comprising a compressible element. The method comprises compressing the compressible element of the bioreactor, detecting a compression force applied to the compressible element and/or a displacement of the compressible element, and determining if the bioreactor has a leak based on the compression force and/or displacement.

In examples, the bioreactor comprises a container and a lid assembly, and the compressible element is an expansion container in fluid communication with the container. The method comprises compressing the compressible expansion container to leak-test the bioreactor.

In accordance with the present disclosure there is also provided a method of detecting a blocked filter of a bioreactor comprising a compressible element, and outlet and a filter arranged in the outlet, the method comprising compressing the compressible element of the bioreactor to urge gas through the outlet, detecting a compression force applied to the compressible element, and determining if the compression force exceeds a predefined threshold indicating that the filter is blocked.

In examples, the method further comprises the step of providing an alarm or an alert to a user in response to determining that the compression force exceeds the predefined threshold. Particularly, upon determination that the compression force exceeds a predefined threshold indicating that the filter is blocked, a signal may be provided to a microprocessor. In such examples, upon receiving the signal at the microprocessor, the microprocessor may provide an (e.g., visual) alert or an (e.g., audible) alarm to a user.

### BRIEF DESCRIPTION OF THE DRAWINGS

Embodiments of the disclosure are further described hereinafter with reference to the accompanying drawings, in which:

FIG. 1 shows a cell processing system that includes a bioreactor;

FIGS. 2A and 2B illustrate the bioreactor of the cell processing system of FIG. 1;

FIG. 3 illustrates a support of the cell processing system of FIG. 1;

FIGS. 4A and 4B illustrate a first example load sensing unit of the cell processing system of FIG. 1;

FIGS. 5A and 5B illustrate a clamp of the load sensing unit of FIGS. 4A and 4B, and a corresponding clamp feature on the bioreactor;

FIG. 6 illustrates a load cell of the load sensing unit of FIGS. 4A and 4B;

FIG. 7 schematically illustrates an alternative example load sensing unit of the cell processing system of FIG. 1;

FIGS. 8A and 8B illustrate an alternative example bioreactor;

FIGS. 9A and 9B illustrate an alternative example load sensing unit for use with the bioreactor of FIGS. 8A and 8B;

FIG. 10 illustrates an alternative example bioreactor;

FIG. 11 illustrates an alternative example load sensing unit for use with the bioreactor of FIG. 10;

FIG. 12 illustrates an alternative example bioreactor;

FIG. 13 illustrates an alternative example load sensing unit for use with the bioreactor of FIG. 12; and

FIG. 14 shows the load sensing unit of FIG. 13 coupled with the bioreactor of FIG. 12.

### DETAILED DESCRIPTION

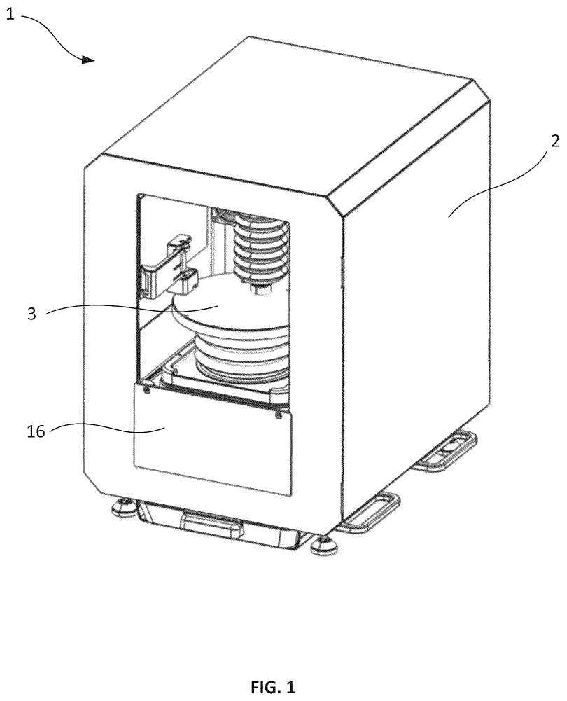

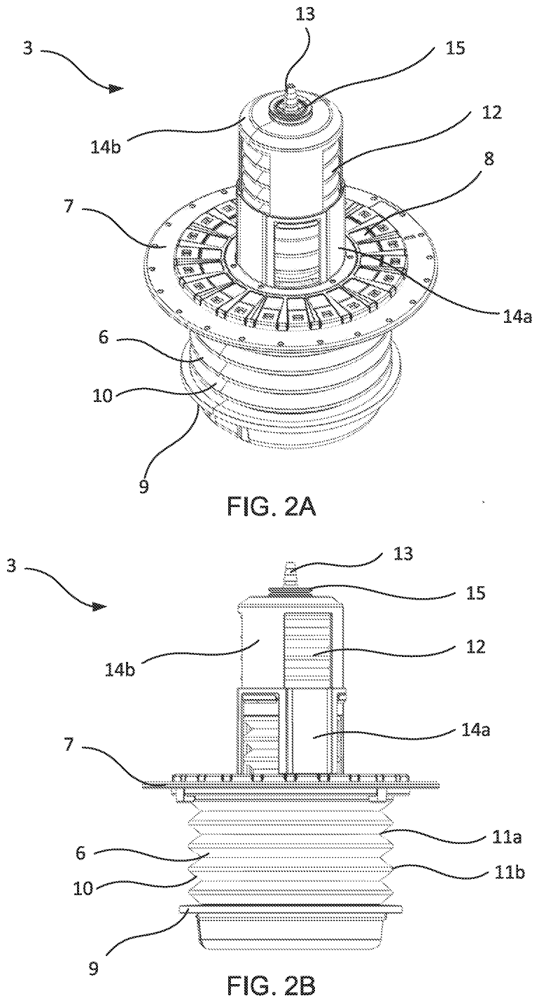

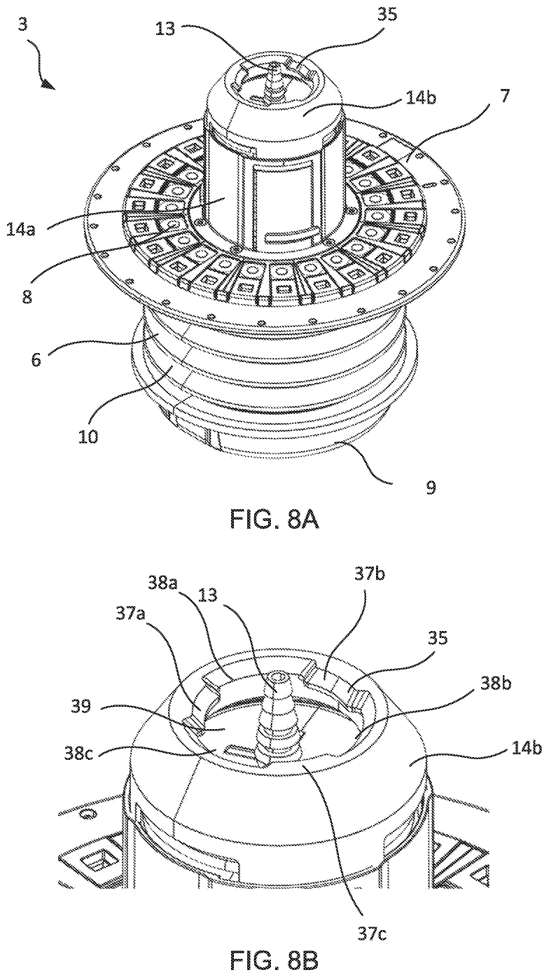

FIG. 1 shows a cell processing system 1 that includes a housing 2 and a bioreactor 3. FIG. 1 shows the bioreactor 3 loaded into the housing 2. FIGS. 2A and 2B illustrate a first example of the bioreactor 3 in isolation, and FIGS. 8A, 8B, 10 and 12 illustrate further examples of the bioreactor 3. During use cells are processed, for example, cultured, within the bioreactor 3 in the housing 2 of the cell processing system 1.

The housing 2 provides a closed environment for the bioreactor 3. The housing 2 is provided with power, connectivity and other utilities needed for the cell processing within the bioreactor 3. The cell processing system 1 includes a temperature control system to control the temperature within the housing 2. The cell processing system 1 includes a humidity control system to control the humidity within the housing 2. The cell processing system 1 includes a gas control system to control gas flow into and out of the housing 2, for example, to control pressure within the housing 2 and/or to control gas concentrations within the housing 2, for example, oxygen and carbon dioxide concentrations. The housing 2 may be an incubator within which the bioreactor 3 is housed during cell processing.

As shown in FIGS. 2A and 2B, the bioreactor 3 comprises a container 6 and an interface plate 7. The interface plate 7 is a lid of the container 6. The interface plate 7 comprises at least one connector interface 8 for connecting to an external component, for example, a consumable that can be temporarily attached to the bioreactor 3. In examples, the or each connector interface 8 includes a septum seal that maintains a sealed environment within the container 6. Access to the container 6 can be provided by a needle that passes through the septum seal of the connector interface 8 to create a fluid connection into the container 6. Accordingly, consumables can be connected to the connector interface 8 to add material to, or remove material from, the bioreactor 3, in particular, the container 6.

The container 6 is a compressible container. In particular, the container 6 has a bottom wall 9 disposed opposite to the interface plate 7, and a compressible wall 10 defining a sidewall of the container 6. The compressible wall 10 extends between, and is attached to, the interface plate 7 and the bottom wall 9. The compressible wall 10 and bottom wall 9 may be integrally formed. The compressible wall 10 is compressible such that the bottom wall 9 can move toward and away from the interface plate 7, changing the internal volume of the container 6. The compressible wall 10 also allows the angle of the bottom wall 9 with respect to the interface plate 7 to be varied, for example, to mix or agitate the contents of the container 6.

The compressible wall 10 is a bellows wall, having a concertina arrangement that allows the compressible wall 10 to fold onto itself in order to collapse. In particular, the compressible wall 10 comprises a series of alternately arranged inward folds 11a and outward folds 11b that allow the compressible wall 10 to collapse like a bellows or concertina. The inward folds 11a and outward folds 11b are formed by thinned sections in the compressible wall 10. The inward folds 11a comprise a thinned section arranged on the outer surface of the compressible wall 10, and the outward folds 11b comprise a thinned section arranged on the inner surface of the compressible wall 10.

The container 6 of the bioreactor 3 can therefore expand and contract, or be expanded and contracted. In particular, the compressible container 6 may expand as the cell culture within the container 6 grows, and/or as additional materials are added, or it may be moved (e.g., compressed or expanded) to change the volume of the container 6. The cell processing system (1, see FIG. 1) may comprise an actuator (not shown) adapted to move, for example, push and/or pull, the bottom wall 9 of the container 6 and/or the interface plate 7 to change the volume of the container 12. The actuator may be operable to agitate the contents of the container 6, for example, by moving the bottom wall 9 in a reciprocal motion. The actuator may be an agitator assembly.

As illustrated in FIGS. 2A and 2B, the interface plate 7 also includes an expansion container 12, otherwise called a breathing bellows. The expansion container 12 is collapsible, for example, being formed of a bellows like the container 6. The expansion container 12 is in fluid communication with the container 6 through an opening in the interface plate 7. The expansion container 12 may comprise a filter 13 that filters air and other gases passing into or out of the expansion container 12. The filter 13 may be closable to seal the expansion container 12. In other examples, the expansion container 12 is closed and sealed from the external environment.

A cage 14 is provided around the expansion container 12 and keeps the expansion container 12 in line as it expands and contracts. As shown in FIGS. 2A and 2B, the cage 14 comprises a first cage part 14a attached to the interface plate 7 and a second cage part 14b slidably mounted to the first cage part 14a. The second cage part 14b can slide in the direction of expansion/contraction of the expansion container 12. A block prevents the second cage part 14b from disengaging from the first cage part 14a.

Accordingly, the expansion container 12 can expand or contract depending on operation and environmental characteristics of the bioreactor 3. As the expansion container 12 expands and contracts the cage 14 constrains movement the expansion container 12 and the first and second cage parts 14a, 14b slide relative to each other.

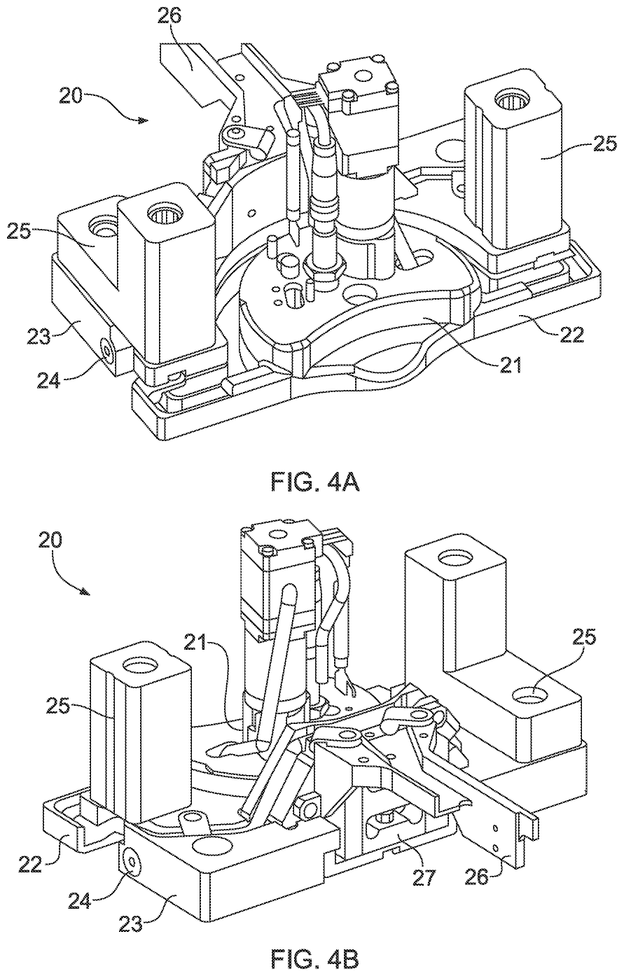

As described below with reference to FIGS. 4A to 6, a coupling is provided to couple the bioreactor 3, in particular, the second cage part 14b, to a load sensing unit. In this example, the coupling comprises a clamp.

As illustrated in FIGS. 2A and 2B, the cage 14 also includes a clamp feature 15, in this example a lip. The clamp feature 15 is grippable by an actuator of a load sensing unit as described further hereinafter. In other examples where the bioreactor 3 does not include an expansion container 12, the lip 15 may be provided on the interface plate 7 or other part of the bioreactor 3.

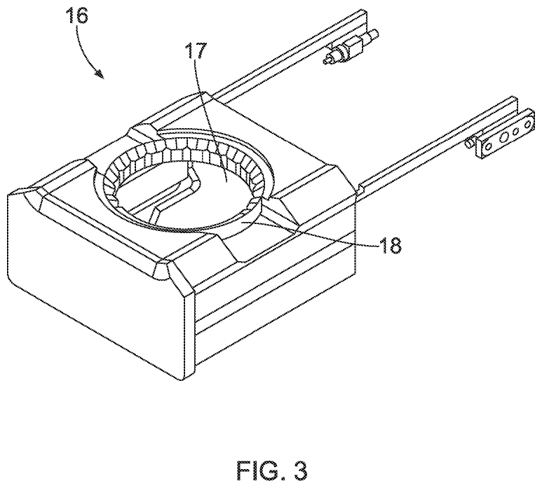

The cell processing system 1 comprises a support 16, as illustrated in FIGS. 1 and 3, mounted within the housing 2 to receive and support the bioreactor 3. The support 16 may slide in and out of the housing 2 in the manner of a drawer for convenience of loading and unloading the bioreactor 3. The support 16 comprises a support portion 18 adapted to engage the interface plate 7 and support the bioreactor 3. The support 16 also includes an opening 17 adapted to receive the container 6 of the bioreactor 3 such that the interface plate 7 rests on the support portion 18 and the container 6 is suspended below, in the opening 17. The support 16 holds the interface plate 7 of the bioreactor 3 in a substantially horizontal position.

Referring to FIGS. 1 to 3, when the bioreactor 3 is supported on the support 16 the expansion container 12 is positioned on the top of the interface plate 7 and is expandable and contractable in a substantially vertical direction. The container 6 is suspended below the interface plate 7 and may be freely hanging, supported on another plate, and/or moved by an actuator or agitator assembly as described above.

In some examples, the bioreactor 3 is rotatable within the housing 2. In such examples the support 16, in particular, the support portion 18, may comprise a rotating mechanism. For example, the support portion 18 may have a pancake motor adapted to rotate the interface plate 7 and therefore rotate the bioreactor 3.

As shown in FIG. 2A, the bioreactor 3 comprises a plurality of connector interfaces 8, and the rotating mechanism may be operable to rotate the interface plate 7 to align successive connector interfaces 8 with another component or assembly, for example, a sampling or material addition consumable. The rotating mechanism may be adapted to index the interface plate 7 to bring successive connector interfaces 8 into alignment with a connector, for example, a connector for a sampling or material addition consumable.

The cell processing system 1 may further include one or more consumables. The consumables may be attachable to the bioreactor 3 and/or to another assembly provided within the housing 2. In particular, one or more consumables may be attached to an actuator that connects the consumable to the bioreactor 3. Alternatively, the one or more consumables may be connected to the bioreactor 3, for example, at the connector interface(s) 8, and the cell processing system 1 may comprise an actuator to operate the consumable. For example, the cell processing system 1 may comprise an actuator adapted to depress or compress a consumable to move a material from the consumable into the container 6, and/or the actuator may be operable to retract or expand the consumable to draw material from the container 6. The cell processing system 1 may include a consumable loading mechanism at which a user loads a consumable into the housing 2. The consumable loading mechanism may then be operated to attach the consumable to the bioreactor 3, for example, at a connector interface 8 as illustrated in FIGS. 2A and 2B.

In examples, the consumables may be connected to the bioreactor 3, in particular, to the connector interface 8 of the interface plate 7, by a common connector. The connector may maintain sterility between the consumable and the bioreactor 3, for example, by having one or more seals such a septum seals. The connector may be that described in patent application PCT/GB2020/053229 (WO2021123760A1).

The cell processing system 1 may additionally include various components and systems that interact with the housing 2, bioreactor 3 and/or consumables. For example, as mentioned above, the housing 2 may include an agitator that acts to agitate the bioreactor 3 so as to agitate a cell suspension provided within the bioreactor 3. In other examples, the cell processing system 1 may include a consumable loading mechanism adapted to hold one or more consumables. In examples, the cell processing system 1 may include an actuator operable to actuate one or more the consumables. The cell processing system 1 may be configured for automated or semi-automated operation, and/or may permit manual operation.

As described above, the bioreactor 3 includes a container 6 and an interface plate 7. During use for cell processing the container 6 holds a fluid in which the cell processing occurs. In particular, the fluid comprises a population of cells present in a liquid medium. The consumables may attach to the bioreactor 3 to add material to the container 6. For example, the consumables may add cells (e.g., a cell suspension), a cell growth media, or other material. The consumables may alternatively attach to the bioreactor 3 to remove material from the container 6. For example, the consumables may remove a waste material, a sample, and/or processed cells. The consumables therefore connect to the bioreactor 3 in order to facilitate process steps of the cell processing.

The population of cells being processed in the bioreactor 3 during use may comprise any cell type. Suitably the population of cells may comprise a homogenous population of cells. Alternatively, the population of cells may comprise a mixed population of cells.

The population of cells may comprise any human or animal cell type, for example: any type of adult stem cell or primary cell, T cells, CAR-T cells, monocytes, leukocytes, erythrocytes, NK cells, gamma delta t cells, tumor infiltrating t cells, mesenchymal stem cells, embryonic stem cells, induced pluripotent stem cells, adipose derived stem cells, Chinese hamster ovary cells, NS0 mouse myeloma cells, HELA cells, fibroblasts, HEK cells, insect cells, organoids etc. Suitably the population of cells may comprise T-cells.

Alternatively, the population of cells may comprise any microorganism cell type, for example: bacterial, fungal, Archaean, protozoan, algal cells.

In examples, a liquid medium may be added to the container 6 during cell processing. The liquid media may be any sterile liquid capable of maintaining cells. The liquid medium may be selected from: saline or may be a cell culture medium. The liquid medium may be a cell culture medium selected from any suitable medium, for example: DMEM, XVIVO 15, TexMACS. The liquid medium may be appropriate for the type of cells present in the population. For example, the population of cells comprises T cells and the liquid medium comprises XVIVO 10.

In examples, the liquid medium may further comprise additives, for example: growth factors, nutrients, buffers, minerals, stimulants, stabilizers or the like.

In examples, the liquid medium comprises growth factors such as cytokines and/or chemokines. The growth factors may be appropriate for the type of cells present in the population and the desired process to be carried out. The liquid medium may comprise stimulants such as antigens or antibodies, which may be mounted on a support. Suitable stimulants are appropriate for the type of cells present in the population and the desired process to be carried out. When culturing T-cells, for example, antibodies are provided as a stimulant in the liquid medium. The antibodies may be mounted on an inert support such as beads, for example: dynabeads.

The additives may be present in the liquid medium at an effective concentration. An effective concentration can be determined by the skilled person on the basis of the population of cells and the desired process to be carried out using known teachings and techniques in the art.

In examples, the population of cells is seeded in the liquid medium at a concentration of between 1×104 cfu/ml up to 1×108 cfu/ml.

FIGS. 4A to 6 illustrate a first example load sensing unit 20 of the cell processing system 1. The load sensing unit 20 is positioned within the housing 2 of the cell processing system 1. In particular, the load sensing unit 20 is mounted above the bioreactor 3 within the housing 2.

As described further hereinafter, in some examples, the load sensing unit 20 is adapted to connect to the bioreactor 3 to detect a weight of the bioreactor 3. In examples, the load sensing unit 20 is adapted to connect to the bioreactor 3 and to lift the bioreactor 3 away from the support portion 18 such that the entire weight of the bioreactor is borne by the load sensing unit 20. In other examples, the load sensing unit 20 may connect to the bioreactor 3 and the support portion 18 may move to disengage from the bioreactor 3 such that the entire weight of the bioreactor is borne by the load sensing unit 20. The load sensing unit 20 may comprise a load cell for detecting a weight of the bioreactor 3.

In other examples, the load sensing unit 20 is adapted to compress a part of the bioreactor, in particular, the expansion container (12, see FIGS. 2A and 2B) and measure a compression force. The load sensing unit 20 may comprise a load cell arranged to detect the compression force. The detected compression force may be used to leak-test the bioreactor 3 and/or to determine if the filter (13, see FIGS. 2A and 2B) is blocked.

As shown in FIGS. 4A and 4B, the load sensing unit 20 comprises a clamp 21, described further hereinafter, operable to clamp onto the bioreactor (3, see FIGS. 2A and 2B). As explained above, the load sensing unit 20 is positioned above the bioreactor 3 within the housing and as shown in FIG. 4A the clamp 21 is downwards facing to clamp onto the top of the bioreactor. In particular, the clamp 21 is operable to clamp onto the clamp feature 15 of the bioreactor 3 as shown in FIGS. 2A and 2B.

The clamp 21 is mounted on a hinge plate 22 that is pivotally connected to a base plate 23 via a pivot. The pivot is provided by shafts 24a, 24b.

The base plate 23 is mountable to the housing 2 of the cell processing system 1 shown in FIG. 1. The base plate 23 includes one or more linear actuators 25 that provide linear movement of the base plate 23 and load sensing unit 20 relative to the housing 2. In particular, the linear actuators 25 permit vertical movement of the base plate 23 within the housing 2 of the cell processing system 1. The base plate 23 can thereby be moved toward and away from the bioreactor 3 within the housing 2.

In use, the clamp 21 can clamp onto the top of the bioreactor (in particular, the clamp feature 15 shown in FIGS. 2A and 2B) and the base plate 23 can be lifted by the linear actuator 25 such that the weight of the bioreactor is borne by the load sensing unit 20. The weight of the bioreactor will urge rotation of the hinge plate 22 about the shafts 24.

As shown in FIG. 4B, a load cell 27 is arranged between the base plate 23 and the hinge plate 22 such that the torque of the hinge plate 22 acts on the load cell 27. In this example, the torque of the hinge plate 22 acts to compress the load cell 27, but in other examples the torque of the hinge plate 22 may act to place the load cell 27 under tensile stress. The load cell 27 includes one or more sensors arranged to detect the load acting on the load cell 27. For example, the load cell 27 may include one or more strain gauges such as a piezoresistive strain gauge, inductive or reluctance strain gauge, or a magnetostrictive strain gauge. Sensor signals output from the load cell sensors can be received at a controller of the cell processing system 1. The controller can be configured to determine a weight of the bioreactor.

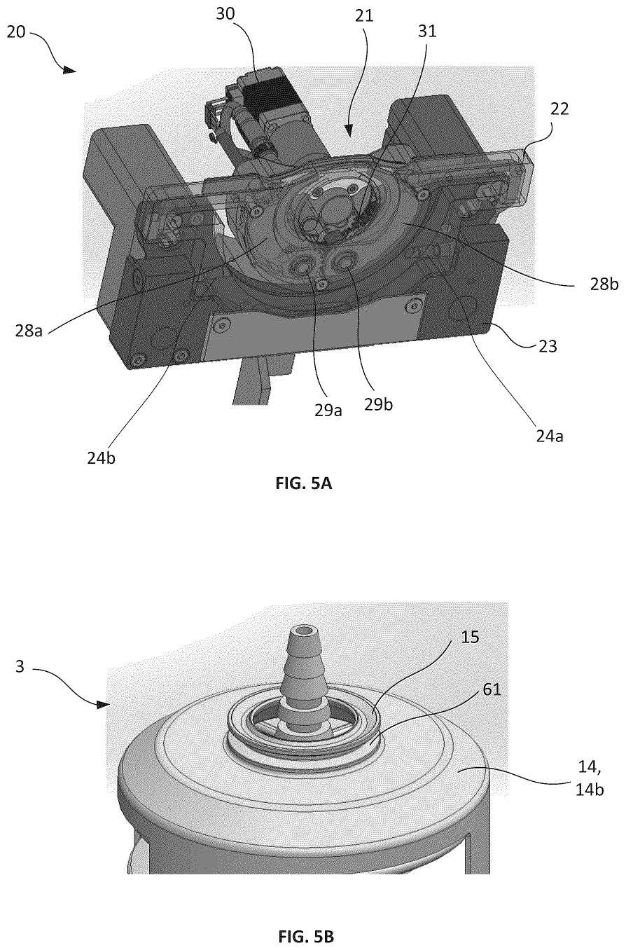

FIGS. 5A and 5B illustrate the clamp 21 and clamping feature 15 on the bioreactor 3 in more detail. As shown in FIG. 5A, the clamp 21 has two clamp arms 28a, 28b that are each pivotally mounted to the hinge plate 22 at pivots 29a, 29b. A motor 30 is provided to actuate the clamp arms 28a, 28b via a gear train 31 that is arranged to translate the rotation output by the motor into opposing rotation of the clamp arms 28a, 28b about the pivots 29a, 29b. In this way, the clamp arms 28a, 28b can be driven to engage or disengage the clamping feature 15 on the top of the bioreactor 3, shown in FIG. 5B. As shown, the clamping feature 15 comprises a lip formed by a depression 61 that receives the clamp arms 28a, 28b, allowing the clamp 21 to be secured to the bioreactor 3. The gear train 31 may include a self-locking feature such that in the clamped position the clamp arms 28a, 28b are held in place and cannot move out of the clamped position unless actuated by the motor 30.

Accordingly, the motor 30 can be driven in one direction to cause the clamp arms 28a, 28b to clamp onto the clamping feature 15, and in the other direction to cause the clamp arms 28a, 28b to release the clamping feature 15.

The load sensing unit 20 may include one or more sensors arranged to detect gripping of the bioreactor (in particular, the clamping feature 15) by the clamp 21, and/or the positions of the clamp arms 28a, 28b to verify that the clamp 21 is connected to the bioreactor 3.

As described above, the load sensing unit 20 is movable, substantially vertically, into and out of a position in which the bioreactor 3 can be clamped. In examples, the clamp 21 may comprise one or more sensors to detect the presence or position of the bioreactor 3 relative to the clamp arms 28a, 28b. For example, a capacitive proximity sensor may be provided to detect the cage 14, clamping feature 15 and/or filter 13 of the bioreactor 3. The one or more sensors may comprise a switch or a proximity sensor, for example, a capacitive proximity sensor. The one or more sensors are activated when the clamp 21 is in the correct position relative to the bioreactor 3 to clamp onto the clamping feature 15. The one or more sensors may communicate sensor signals with a controller configured to control the motor 30 to close the clamp 21 when the clamp 21 is in the correct position.

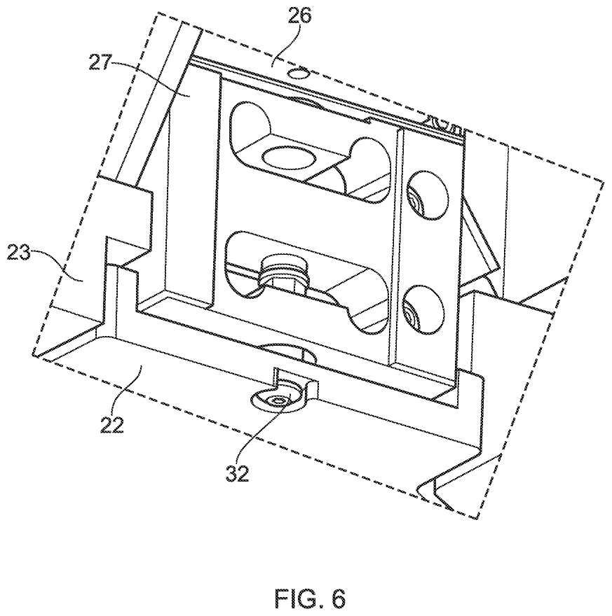

FIG. 6 shows the load cell 27 in more detail. As shown, the load cell 27 is positioned between the hinge plate 22 and the base plate 23, in this example between the hinge plate 22 and a mounting arm 26 that is attached to the base plate 23. A load pin 32 connects the hinge plate 22 to the load cell 27. During use, when the weight of the bioreactor is borne by the load sensing unit 20 the torque of the hinge plate 22 acts to compress the load cell 27 against the base plate 23. A strain imparted on the load cell 27 can be detected by a sensor to determine the weight of the bioreactor.

In some examples, the load pin 32 is threaded and threadingly attached to both the hinge plate 22 and the base plate 23 (or mounting arm 26). In this example the hinge plate 22 is spaced from the load cell 27. In this way, as the bioreactor is lifted, the weight of the bioreactor urges rotation of the hinge plate 22 and applies a torque to the load cell 27 via the load pin 32.

Accordingly, a first method is provided for determining the weight of the bioreactor 3. In this method, the load sensing unit 20 is positioned and clamped onto the bioreactor 3 to connect the load sensing unit 20 to the bioreactor 3. The load sensing unit 20 is then lifted such that the bioreactor 3 is also lifted and the entire weight of the bioreactor 3 is borne by the load sensing unit 20. The force applied to the load cell 27 can be determined and used to determine a weight of the bioreactor 3.

In some examples, the load sensing unit 20 is operable to apply a compressive force on the bioreactor 3. In particular, the linear actuator 25 may be operated to move the base plate 23 and hinge plate 22 toward the bioreactor 3, to compress the bioreactor 3, in particular, the expansion container 12 and/or container 6 of the bioreactor as described with reference to FIGS. 2A and 2B. As the bioreactor 3 is compressed the load cell 27 will be placed under strain and the strain force can be detected by the load cell 27. In this example the load pin 32 is threaded to both the hinge plate 22 and load cell 27 such that the torque is entirely transferred.

In this example, the cell processing system 1 may additionally detect the displacement of the load sensing unit 20 as the bioreactor 3 is compressed. For example, the linear actuators 25 may include an encoder (e.g., a digital encoder) to detect the displacement of the base plate 23. Alternatively, a separate sensor may detect displacement of the base plate 23. From the detected displacement and/or force detected by the load cell 27, a controller may be configured to determine if the bioreactor 3 has a leak. That is, the load sensing unit 20 may be operated to compress the bioreactor 3 in order to leak-test the bioreactor 3.

Accordingly, a second method is provided for leak-testing the bioreactor 3. In this method, the load sensing unit 20 is moved to compress the bioreactor 3. In particular, the load sensing unit 20 is moved to compress the expansion container 12 of the bioreactor 3. The compressive force applied to the bioreactor is detected as strain on the load cell 27. A displacement of the load sensing unit 20 is also detected. Based on the compressive force and displacement a controller can determine if the bioreactor 3 has a leak.

In some examples, the load sensing unit 20 may be used to test whether the filter 13 (embedded in the expansion container 12 as shown in FIGS. 2A and 2B) is blocked. In this example, the method comprises first coupling the load sensing unit 20 to the bioreactor 3 as described above. Then, the load sensing unit 20 is moved to compress the bioreactor 3, in particular, the expansion container 12. At the same time, the compressible container 6 is also compressed by another actuator of the cell processing system 1, which would urge gases from the container 6 into the expansion container 12. Usually, this gas would exit through the filter 13, but if the filter 13 is blocked then pressure would build up within the bioreactor 3 and the load cell 27 would detect the increased pressure. The load cell 27 can be calibrated to determine a detected pressure when the gas flow rate through the filter 13 is normal (i.e., filter 13 is not blocked), and so that a control unit that receives a signal from the load cell 27 can determine when the filter 13 is blocked (or partially blocked). The control unit includes a microprocessor to receive such a signal and, upon receiving a signal from the load cell 27 indicating that the filter 13 is partially or fully blocked, may provide a visual alert or audible alarm to an operator of the load sensing unit 20.

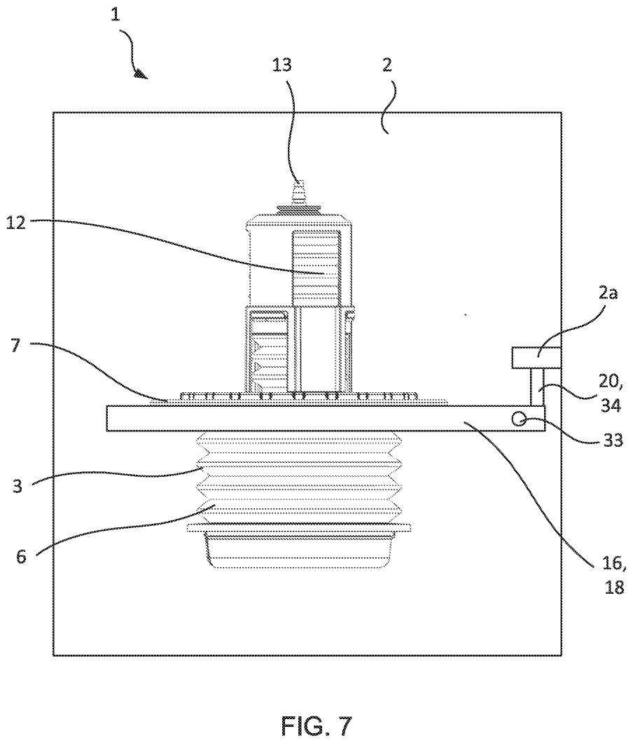

FIG. 7 schematically illustrates an alternative example cell processing system 1. As shown, the cell processing system 1 includes a housing 2 within which the bioreactor 3 is received. The housing 2 is the same as described with reference to FIG. 1. The support 16, in particular, the support portion 18, supports the bioreactor 3. In particular, as shown in FIG. 3, the support 18 includes a support portion 18 on which the interface plate 7 of the bioreactor 3 is supported. As shown in FIG. 7, in this example the support portion 18 is pivotally mounted to the housing 2 at pivot 33. Accordingly, the weight of the bioreactor 3 generates a torque about the pivot 33. A load sensing unit 20 of this example comprises a load cell 34 disposed between the support portion 18 and a part 2a of the housing 2 such that the load cell 34 is compressed by the torque of the support portion 18. In this way, similar to the above examples, the weight of the bioreactor 3 can be determined based on the torque detected by the load cell 34. In other examples, the load cell 34 may be arranged to be placed under tensile stress by the weight of the bioreactor 3 on the support portion 18.

In the example of FIG. 7, a method of detecting a weight of the bioreactor 3 comprises disengaging any other parts of the cell processing system 1 from the bioreactor 3. For example, any actuators, agitation mechanisms, etc., are disconnected from the bioreactor 3 so that only the weight of the bioreactor 3 is acting on the support portion 18. In this state the load detected by the load cell 34 can be used to determine a weight of the bioreactor 3. In some examples, a consumable may be attached to the bioreactor 3 during the method of detecting the weight of the bioreactor 3. The consumable may have a known weight that can be accounted for. An advantage of the example of FIG. 7 is that the load sensing unit 20 does not need to clamp onto or release the bioreactor 3 during use, and the weight of the bioreactor 3 may be constantly monitored.

In the example of FIG. 7, an actuator (not shown) within the housing 2 may be operable to compress the expansion container 12 of the bioreactor 3. The actuator can act on the expansion container 12 to compress the expansion container 12 against the support 18. As the actuator compresses the expansion container 12 the compression force can be detected at the load cell 34. In addition, a displacement of the expansion container 12 can be detected, for example, by detecting a displacement of the actuator. Accordingly, the bioreactor 3 can be leak-tested and/or a blocked filter 13 can be detected in the same way as described for previous examples.

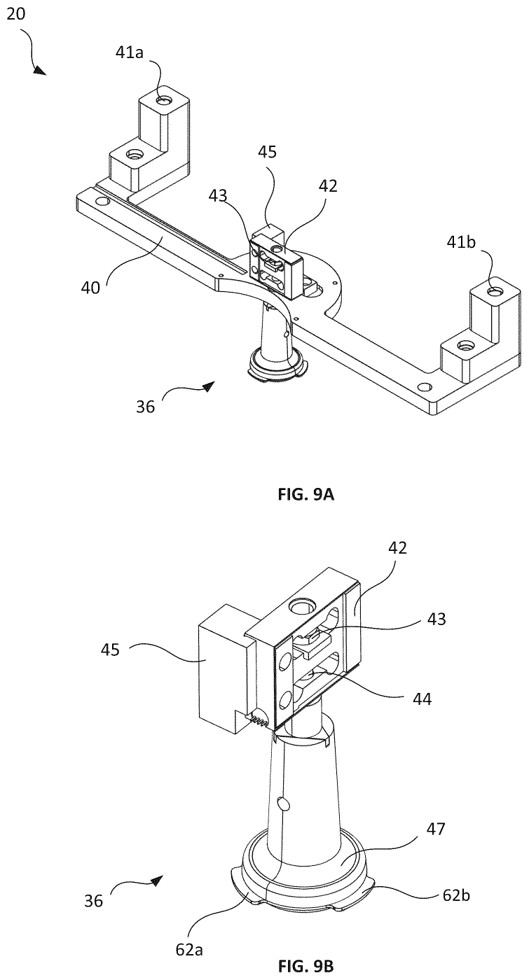

FIGS. 8A to 9B Illustrate a further example cell processing system, in particular, a bioreactor 3 and a load sensing unit 20. The bioreactor 3 is substantially the same as described above with reference to FIGS. 2A and 2B, except for the coupling as described further below. In particular, the bioreactor 3 has a container 6 and an interface plate 7. The interface plate 7 comprises at least one connector interface 8 for connecting to an external component. The container 6 is a compressible container, having a bottom wall 9 and a compressible wall 10 extending between the interface plate 7 and the bottom wall 9. The bioreactor 3 also includes an expansion container (not visible in FIGS. 8A to 10) and a cage 14 surrounding the expansion container. The expansion container 12 may comprise a filter 13.

The load sensing unit 20 is positioned within the housing 2 of the cell processing system 1 shown in FIG. 1. In particular, the load sensing unit 20 is mounted above the bioreactor 3 within the housing 2.

As with the previous examples, the load sensing unit 20 is adapted to connect to the bioreactor 3 to detect a weight of the bioreactor 3. In examples, the load sensing unit 20 is adapted to connect to the bioreactor 3 and to lift the bioreactor 3 away from the support portion 18 such that the entire weight of the bioreactor is borne by the load sensing unit 20. In other examples, the load sensing unit 20 may connect to the bioreactor 3 and the support portion 18 may move to disengage from the bioreactor 3 such that the entire weight of the bioreactor is borne by the load sensing unit 20. The load sensing unit 20 may comprise a load cell for detecting a weight of the bioreactor 3.

In other examples, the load sensing unit 20 is adapted to compress a part of the bioreactor, in particular, the expansion container (12, see FIGS. 2A and 2B) and measure a compression force. The load sensing unit 20 may comprise a load cell arranged to detect the compression force. The detected compression force may be used to leak-test the bioreactor 3 and/or to determine if the filter 13 is blocked.

FIGS. 8A to 9B illustrate the coupling between the load sensing unit 20 and bioreactor 3, in particular, the second cage part 14b. In this example, as described below, the coupling is a bayonet coupling.

As shown in FIGS. 8A and 8B, in this example the cage 14 of the bioreactor 3 includes a first bayonet fitting 35. An end of the cage 14, in particular, the second cage part 14b, includes an opening 39. The first bayonet fitting 35 is formed in the opening 39. The first bayonet fitting 35 includes three recesses 38a-38c separated by lugs 37a-37c. The lugs 37a-37c extend into the opening 39. In other examples the first bayonet fitting 35 may comprise a different number of recesses 38 and lugs 37, for example, two, four, or five.

In other examples where the bioreactor 3 does not include an expansion container 12, the first bayonet fitting 35 may be provided on the interface plate 7 or other part of the bioreactor 3.

The first bayonet fitting 35 can be engaged by a corresponding second bayonet fitting 36 on the load sensing unit 20, shown in FIGS. 9A and 9B.

As shown in FIG. 9A, the load sensing unit 20 includes a platform 40 that is mounted in the housing 2 of the cell processing system 1 above the bioreactor 3 (see FIG. 1). The platform 40 is mounted on linear bearings 41a, 41b and can be moved, by an actuator, toward and away from the bioreactor 3 (i.e., up and down).

The second bayonet fitting 36 is mounted to the platform 40 via load cell 42. A threaded section 44 (e.g., a bolt) extends from the second bayonet fitting, through an opening in the platform 40, and into the load cell 42 where it is secured by nut 43. A bracket 45 mounts the load cell 42 to the platform 40.

The load cell 42 may include one or more strain gauges such as a piezoresistive strain gauge, inductive or reluctance strain gauge, or a magnetostrictive strain gauge. Sensor signals output from the load cell sensors can be received at a controller (control unit) of the cell processing system 1. The controller (control unit), particularly a microprocessor thereof, can be configured to determine a weight of the bioreactor and provide visual alerts and/or audible alarms to a user.

In this way, when the second bayonet fitting 36 couples to the first bayonet fitting 35 and the platform 40 is raised to lift the bioreactor, the load cell 42 will detect a weight of the bioreactor.

As shown in FIG. 9B, the second bayonet fitting 36 comprises three lugs 46a-46c (only two visible in FIG. 9B) extending radially from a hub 47. The lugs 46a-46c are sized to fit through the recesses 38a-38c of the first bayonet fitting 35 (see FIGS. 8A and 8B). After relative rotation of the first bayonet fitting 35 and the second bayonet fitting 36, the lugs 37a-37c of the first bayonet fitting 35 and the lugs 46a-46c of the second bayonet fitting 36 are aligned and this couples the load sensing unit 20 to the bioreactor 3.

As explained above, the bioreactor 3 is rotatable within the housing 2, for example, within the support 16 shown in FIG. 3. In order to couple and decouple the first and second bayonet fittings 35, 36, the vertical movement can be provided by the platform 40, and the rotation can be provided by rotating the bioreactor 3 (and therefore the first bayonet fitting 35). In this way, the load sensing unit 20 can be coupled to, and decoupled from, the bioreactor 3 using the first and second bayonet fittings 35, 36. In other examples, at least a part of the second bayonet fitting 36 may be rotatable for coupling and decoupling with the first bayonet fitting 35.

When coupled, the platform 40 can be lifted to also lift the bioreactor 3, and the load cell 42 can detect a weight of the bioreactor 3. In other examples, the platform 40 may be driven down, toward the bioreactor 3, and the load cell 42 can detect a force applied to the bioreactor to perform a leak test and/or to detect a blocked filter as described above.

The load sensing unit 20 may include one or more sensors arranged to detect coupling with the bioreactor (in particular, engagement of the first and second bayonet fittings 35, 36).

As described above, the load sensing unit 20 is movable, substantially vertically, into and out of a position in which the bioreactor 3 and load sensing unit 20 can be coupled. In examples, the second bayonet fitting 36 may comprise one or more sensors to detect the presence or position of the bioreactor 3 relative to the lugs 46a-46c. For example, a capacitive proximity sensor may be provided to detect the cage 14, or lugs 37a-37c, and/or the filter 13 of the bioreactor 3. The one or more sensors may comprise a switch or a proximity sensor, for example, a capacitive proximity sensor. The one or more sensors are activated when the second bayonet fitting 36 is in the correct position relative to the bioreactor 3 to couple. The one or more sensors may communicate sensor signals with a controller configured to control the rotation of the bioreactor 3 and/or the second bayonet fitting 36 and/or the movement of the platform 40.

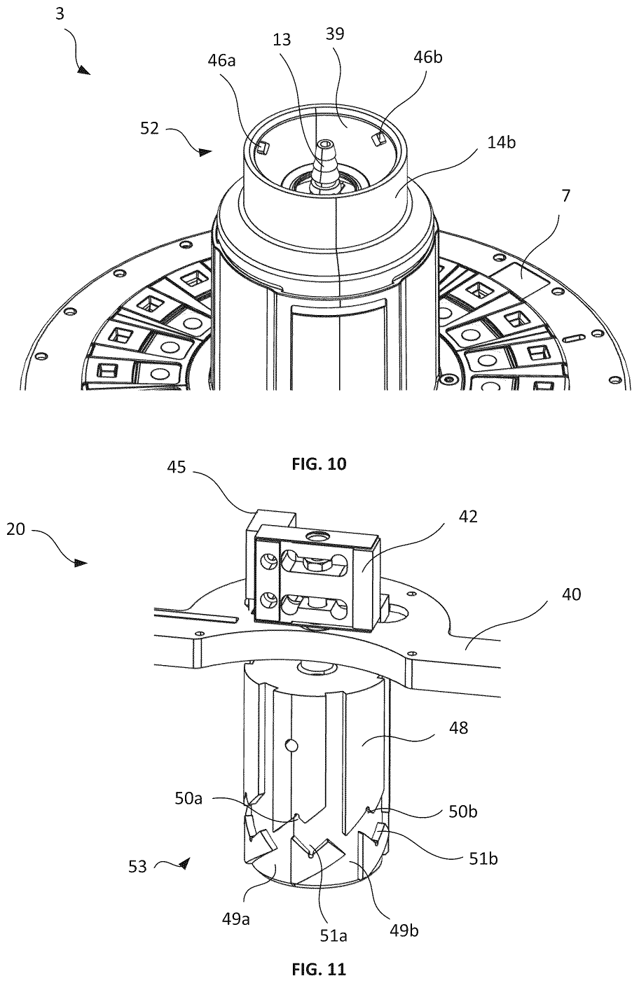

FIGS. 10 and 11 illustrate an alternative example coupling between the load sensing unit 20 and the bioreactor 3. In this example, the coupling is an alternative bayonet coupling, described further below, similar to that of FIGS. 8A to 9B. In particular, FIG. 10 shows an alternative bioreactor 3. The bioreactor 3 is as described with reference to FIGS. 2A and 2B and FIGS. 8A and 8B, except for the coupling, which is a first bayonet fitting 52. FIG. 11 shows an alternative load sensing unit 20 with a second bayonet fitting 53 mounted to the platform 40, which is otherwise as described with reference to FIG. 9A.

As shown in FIG. 10, the cage, in particular, the second cage part 14b, includes an opening 39 and a first bayonet fitting 52 is formed within the opening 39. The first bayonet fitting 52 comprises three inwardly projecting pins 62a-62c (only two are visible in FIG. 10). In this example, there are three pins 62a-62c, but there may be a different number of pins 46a-46c, for example, two, four, or five. In this example, the pins 62a-62c have a square cross-section, but they may be circular or triangular or other shape. The pins 62a-62c are arranged so that a corner of each pin 46a-46c is directed vertically upwards, and an opposite corner of the pin 46a-46c is directed vertically downwards.

As shown in FIG. 11, a second bayonet fitting 53 is provided on the platform 40, which is the same as described with reference to FIG. 9A except for the second bayonet fitting 53. The second bayonet fitting 53 comprises a body, in this example a cylindrical body 48. In other examples the body may have a different shape, such as with a square or hexagonal cross-section. The cylindrical body 48 is mounted to the platform 40 via a load cell 42 and a bracket 45.

The cylindrical body 48 includes grooves 49a, 49b formed on its circumferential surface. The grooves 49a, 49b are spaced to align with the pins 62a-62c of the first bayonet fitting 52 when the cylindrical body 48 is lowered into the opening 39 of the first bayonet fitting 52. There may be a corresponding number of grooves 49a, 49b and pins 62a-62c, or there may be more grooves 49a, 49b than pins 62a-62c but with a circumferential spacing that allows each pin 46a-46c to engage a groove 49a, 49b.

Each groove 49a, 49b includes a first trap 50a, 50b, which faces downwards (toward the bioreactor). The first traps 50a, 50b are formed by angled sides of the grooves 49a, 49b that define a converging point in which a pin 46 will sit when the platform 40 is moved toward the bioreactor 3. When the platform 40 is lowered toward the bioreactor 3 the pins 62a-62c will locate in the first traps 50a, 50b and allow force transfer from the platform 40 to the second cage part 14b for compressing the bioreactor 3.

Each groove 49a, 49b also includes a second trap 51a, 51b, which faces upwards (away from the bioreactor). The second traps 51a, 51b are formed by angled sides of the grooves 49a, 49b that define a converging point in which a pin 46 will sit when the platform 40 is moved away from the bioreactor 3. When the bioreactor 3 is rotated relative to the load sensing unit 20 and the platform 40 is raised away from the bioreactor 3 the pins 62a-62c will sit in the second traps 51a, 51b and allow force transfer from the platform 40 to the second cage part 14b to lift the second cage part 14b and bioreactor 3.

During coupling and decoupling, the bioreactor 3 (and thus also the pins 62a-62c) may be rotated to align with the grooves 49a, 49b, first traps 50a, 50b, and second traps 51a, 51b as needed. Alternatively, the cylindrical body 48 may be rotatably mounted to the platform 40 and the angled sides of the grooves 49a, 49b may cooperate with the pins 62a-62c to cause rotation of the cylindrical body 48 and alignment of the pins 62a-62c and the first and second traps 50a, 50b, 51a, 51b. An actuator may rotate the cylindrical body 48, or it may be freely rotatable.

In this way, the first bayonet fitting 52 and the second bayonet fitting 53 can be used to couple and decouple the load sensing unit 20 and the bioreactor 3. When coupled, the platform 40 can be lifted to also lift the bioreactor 3, and the load cell 42 can detect a weight of the bioreactor 3. In other examples, the platform 40 may be driven down, toward the bioreactor 3, and the load cell 42 can detect a force applied to the bioreactor to perform a leak test and/or to detect a blocked filter as described above.

The load sensing unit 20 may include one or more sensors arranged to detect coupling with the bioreactor (in particular, engagement of the first and second bayonet fittings 52, 53).

As described above, the load sensing unit 20 is movable, substantially vertically, into and out of a position in which the bioreactor 3 and load sensing unit 20 can be coupled. In examples, the second bayonet fitting 53 may comprise one or more sensors to detect the presence or position of the bioreactor 3 relative to the grooves 49a, 49b. For example, a capacitive proximity sensor may be provided to detect the cage 14, or pins 62a-62c, and/or the filter 13 of the bioreactor 3. The one or more sensors may comprise a switch or a proximity sensor, for example, a capacitive proximity sensor. The one or more sensors are activated when the second bayonet fitting 53 is in the correct position relative to the bioreactor 3 to couple. The one or more sensors may communicate sensor signals with a controller configured to control the rotation of the bioreactor 3 and/or the second bayonet fitting 53 and/or the movement of the platform 40.

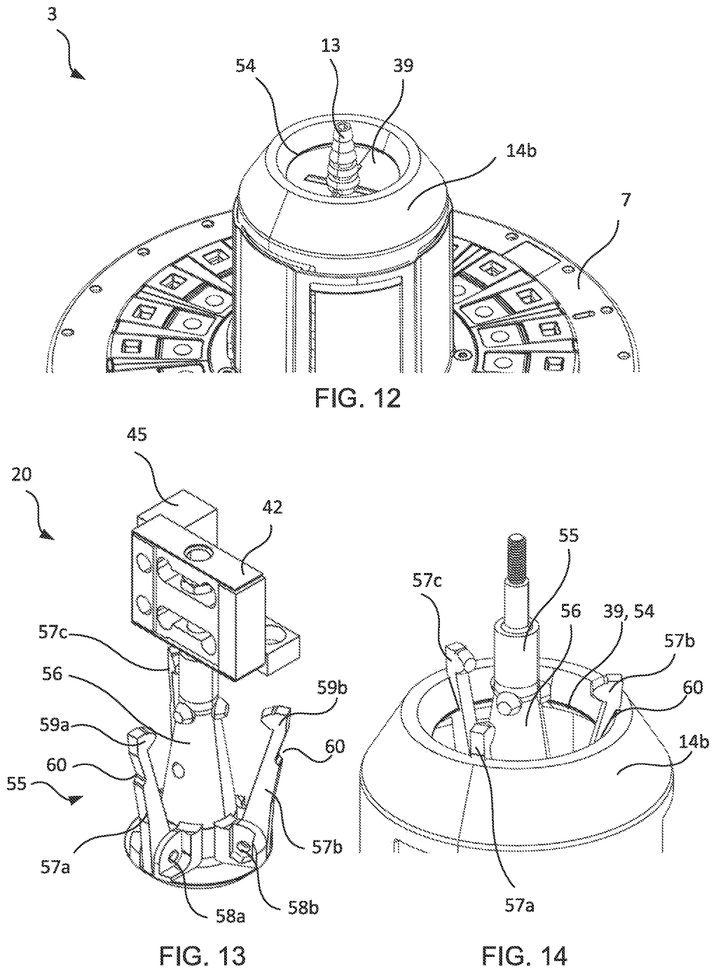

FIGS. 12 to 14 illustrate a further example coupling between the bioreactor 3, in particular, the second cage part 14b, and the load sensing unit 20.

FIGS. 12 to 14 illustrate an alternative example coupling between the load sensing unit 20 and the bioreactor 3. In this example, the coupling is a coupling unit 55 having spring arms 57a-57c, described further below. In particular, FIG. 12 shows an alternative bioreactor 3. The bioreactor 3 is as described with reference to FIGS. 2A and 2B and FIGS. 8A, 8B, and 10, except for the coupling unit 55. FIG. 13 shows an alternative load sensing unit 20 with a coupling unit 55. The coupling unit 55 is mounted to the platform 40 as described with reference to FIG. 9A.

As shown in FIG. 12, in this example the second cage part 14b includes an opening with an inwardly facing edge 54. The edge 54 is circular.

As shown in FIG. 13, the load sensing unit 20 includes a coupling unit 55 that includes a hub 56 and three spring arms 57a-57c. The hub 56 is mounted to the platform 40 (see FIG. 9A) via a load cell 42 and a bracket 45.

The spring arms 57a-57c are pivotally connected to the hub 56 at pivots 58a-58c, and extend upwards (toward the platform (not illustrated) and away from the bioreactor 3). The spring arms 57a-57c each have an end 59a-59c. A torsion spring may be provided at the pivots 58a-58c to urge the spring arms 57a-57c away from the hub 56. Alternatively, helical springs may be provided between the end 59a-59c of each spring arm 57a-57c and the hub 56 to urge the spring arms 57a-57c away from the hub 56.

Each spring arm 57a-57c includes a notch 60 on its outer surface. The notch 60 is formed by two angled sides that form an obtuse angle. As shown in FIG. 14, when the coupling unit 55 is pushed into the opening 39 in the second cage part 14b the spring arms 57a-57c are deflected inwardly until the notches 60 engage the edge 54 of the opening 39. The spring arms 57a-57c are urged outwardly, pressing the notches 60 against the edge 54 of the opening. In this position, the load sensing unit 20 is coupled to the bioreactor 3 and the platform (40, see FIG. 9A) can be raised to lift the bioreactor 3. In addition, the notches 60 are configured such that the coupling unit 55 cannot move relative to the second cage part 14b further into the opening 39, so that the load sensing unit 20 can also press down on the bioreactor 3.

To decouple the coupling unit 55 from the second cage part 14b the coupling unit 55 can be raised up (by raising the platform 40, see FIG. 9A) while the bioreactor 3 is held down (e.g., within the support 16 shown in FIG. 3). The angle of each of the notches 60 will deflect the spring arms 57a-57c inwards and allow the coupling unit 55 to decouple from the opening 39.

In this way, the coupling unit 55 can be used to couple and decouple the load sensing unit 20 and the bioreactor 3. When coupled, the platform 40 can be lifted to also lift the bioreactor 3, and the load cell 42 can detect a weight of the bioreactor 3. In other examples, the load sensing unit 20 can push down on the second cage part 14b, toward the bioreactor 3, and the load cell 42 can detect a force applied to the bioreactor 3 to perform a leak test and/or to detect a blocked filter 13 as described above.

The load sensing unit 20 may include one or more sensors arranged to detect coupling with the bioreactor (in particular, engagement of the coupling unit 55 and the opening 39).

As described above, the load sensing unit 20 is movable, substantially vertically, into and out of a position in which the bioreactor 3 and load sensing unit 20 can be coupled. In examples, the coupling unit 55 may comprise one or more sensors to detect the presence or position of the bioreactor 3 relative to the spring arms 57a-57c. For example, a capacitive proximity sensor may be provided to detect the cage 14, or edge 54, and/or the filter 13 of the bioreactor 3. The one or more sensors may comprise a switch or a proximity sensor, for example, a capacitive proximity sensor. The one or more sensors are activated when the coupling unit 55 is in the correct position relative to the bioreactor 3 to couple. The one or more sensors may communicate sensor signals with a controller configured to control the rotation of the bioreactor 3 and/or the movement of the platform 40.

Throughout the description and claims of this disclosure, the words “comprise” and “contain” and variations of them mean “including but not limited to,” and they are not intended to (and do not) exclude other components, integers or steps. Throughout the description and claims of this disclosure, the singular encompasses the plural unless the context otherwise requires. In particular, where the indefinite article is used, the disclosure is to be understood as contemplating plurality as well as singularity, unless the context requires otherwise.

Features, integers, characteristics or groups described in conjunction with a particular aspect, embodiment or example of the disclosure are to be understood to be applicable to any other aspect, embodiment or example described herein unless incompatible therewith. All of the features disclosed in this disclosure (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The invention extends to any novel one, or any novel combination, of the features disclosed in this disclosure (including any accompanying claims, abstract and drawings), or to any novel one, or any novel combination, of the steps of any method or process so disclosed.

1. A cell processing system for housing a bioreactor, the cell processing system being adapted to support the bioreactor and comprising a load sensing unit operable to detect a weight of the bioreactor.

2. The cell processing system of claim 1, further comprising a housing and a support that supports the bioreactor within the housing.

3. The cell processing system of claim 2, wherein the support is pivotally mounted to the housing and the load sensing unit comprises a load cell positioned relative to the support to receive a torque of the support thereon.

4. The cell processing system of claim 2, wherein the load sensing unit is adapted to releasably connect to the bioreactor.

5. The cell processing system of claim 4, wherein, during use, the load sensing unit is disposed above the bioreactor and operable to releasably connect to a top of the bioreactor.

6. The cell processing system of claim 4, wherein the load sensing unit is movable to lift the bioreactor from the support to a raised position in which the weight of the bioreactor is borne by the load sensing unit.

7. The cell processing system of claim 4, wherein the support is movable to disengage the bioreactor, weight of the bioreactor is borne by the load sensing unit while the support is disengaged from the bioreactor.

8. The cell processing system of claim 4, wherein the load sensing unit comprises a coupling operable to releasably connect to the bioreactor.

9. The cell processing system of claim 8, wherein the coupling comprises a clamp operable to releasably connect to the bioreactor.

10. The cell processing system of claim 9, wherein the clamp comprises an actuator and at least one arm moveable by the actuator to clamp onto the bioreactor.

11. The cell processing system of claim 4, wherein the load sensing unit comprises a pivotally mounted hinge plate, the hinge plate being releasably connectable to the bioreactor, the weight of the bioreactor imparts a torque on the hinge plate while the hinge plate is engaged with the bioreactor.

12. The cell processing system of claim 10, wherein the load sensing unit comprises a load cell arranged to receive the torque imparted on the hinge plate.

13. The cell processing system of claim 8, wherein the coupling comprises a bayonet fitting.

14. The cell processing system of claim 13, wherein the bioreactor comprises a first bayonet fitting and the load sensing unit comprises a second bayonet fitting that is couplable to the first bayonet fitting.

15. The cell processing system of claim 13, further comprising an actuator for rotating the bioreactor relative to the load sensing unit for coupling the first and second bayonet fittings.

16. The cell processing system of claim 8, wherein the coupling comprises at least one spring arm.

17. The cell processing system of claim 16, wherein the load sensing unit comprises a plurality of spring arms arranged to engage an edge of an opening in the bioreactor to couple the load sensing unit to the bioreactor.

18. The cell processing system of claim 17, wherein each of the plurality of spring arms comprises a notch arranged to engage the edge of the opening of in the bioreactor.

19. The cell processing system of claim 1, wherein the bioreactor comprises a compressible element, and wherein the load sensing unit is adapted to detect a compression force as the compressible element is compressed.

20. The cell processing system of claim 19, wherein the load sensing unit comprises an actuator operable to compress the compressible element.

21. The cell processing system of claim 19, further comprising a sensor configured to detect a displacement of the compressible element as the compressible element is compressed.

22. The cell processing system of claim 1, further comprising a bioreactor.

23. The cell processing system of claim 22, wherein the bioreactor comprises a container and a lid assembly having one or more connector interfaces for accessing the internal volume of the container.

24. A method of detecting a weight of a bioreactor within a cell processing system adapted to support the bioreactor, the method comprising bearing the weight of the bioreactor on a load sensing unit of the cell processing system, and determining a weight of the bioreactor based on a load detected by the load sensing unit.

25. The method of claim 24, further comprising releasably connecting the load sensing unit to the bioreactor.

26. The method of claim 25, further comprising connecting the load sensing unit to a lid assembly of the bioreactor.

27. The method of claim 25, further comprising moving the load sensing unit to lift the bioreactor from a support of the cell processing system.

28. The method of claim 25, wherein the bioreactor comprises a compressible element, and wherein the method further comprises moving the load sensing unit to compress the compressible element.

29. The method of claim 28, further comprising detecting a compression force as the load sensing unit compresses the compressible element of the bioreactor.

30. The method of claim 28, further comprising detecting a displacement of the load sensing unit as the load sensing unit compresses the compressible element of the bioreactor.

31. A method of detecting a leak in a bioreactor comprising a compressible element, the method comprising:
compressing the compressible element of the bioreactor;
detecting at least one measurement chosen from among:
a compression force applied to the compressible element; and/or
a displacement of the compressible element; and
determining if the bioreactor has a leak based on the at least one measurement.

32. The method of claim 31, wherein the bioreactor comprises a container and a lid assembly, and wherein the compressible element is an expansion container in fluid communication with the container.

33. A method of detecting a blocked filter of a bioreactor comprising a compressible element, and outlet and a filter arranged in the outlet, the method comprising:
compressing the compressible element of the bioreactor to urge gas through the outlet,
detecting a compression force applied to the compressible element; and
determining if the compression force exceeds a predefined threshold indicating that the filter is blocked.

34. A method of claim 33, further comprising providing an alarm or an alert to a user in response to determining that the compression force exceeds the predefined threshold.
